(19) United States
(12) Patent Application Publication
(10) Pub. No.: US 2022/0042118 A1
(43) Pub. Date: Feb. 10, 2022

(54) METHOD FOR THE RAPID DIAGNOSIS OF COVID-19 USING A MOBILE GENETIC TESTING LABORATORY

(71) Applicant: Machaon Diagnostics, Inc., Oakland, CA (US)

(72) Inventors: James S. Kain, Oakland, CA (US); Michael P. Ero, Oakland, CA (US); Frances Tran, Oakland, CA (US)

(73) Assignee: Machaon Diagnostics, Inc., Oakland, CA (US)

(21) Appl. No.: 17/395,242

(22) Filed: Aug. 5, 2021

Related U.S. Application Data
(60) Provisional application No. 63/061,670, filed on Aug. 5, 2020.

(51) Int. Cl. C12Q 1/70

(52) U.S. Cl. CPC C12Q 1/701

(57) ABSTRACT

The present invention is directed to a method of diagnosing infection with SARS-CoV-2. The method involves first obtaining a sample from a patient, followed by the use of a mobile testing laboratory to test the sample for SARS-CoV-2 nucleic acid. The sample is tested by extracting the RNA from the patient sample, amplifying the nucleic acid using reverse transcription loop-mediated isothermal amplification (RT-LAMP), and measuring the results. Results from the RT-LAMP are obtained within 60 minutes after sample collection, where a positive result is indication of infection with SARS-CoV-2.

### CROSS-REFERENCE TO RELATED APPLICATIONS

The present application claims the benefit of U.S. Provisional Patent Application No. 63/061,670 titled “Method for the Rapid Diagnosis of COVID-19 Using a Mobile Genetic Testing Laboratory,” filed on Aug. 5, 2020, the contents of which are incorporated in this disclosure by reference in its entirety.

### SEQUENCE LISTING

The present application is being filed along with a Sequence Listing in electronic format. The Sequence Listing is provided as a file titled “20210804 sequence listing,” created Aug. 4, 2021, and is 4,000 bytes in size. The information in the electronic format of the Sequence Listing is incorporated herein by reference in its entirety.

### BACKGROUND

Severe acute respiratory syndrome (SARS) is an acute infectious disease that spreads mainly via the respiratory route. SARS can be caused by a coronavirus (SARS-CoV or SARS-CoV-1), which is an enveloped, positive-sense, single-stranded RNA virus which infects the epithelial cells within the lungs. A similar coronavirus, SARS-CoV-2, has been identified as the etiological agent that causes coronavirus disease 2019 (COVID-19).

The current COVID-19 testing environment is not working. The current average turnaround time is 2-4 days for test results, which could lead to consumption of scarce personal protective equipment (PPE) and could result in additional infections. Furthermore, site-based testing puts everyone at risk by having the people who need testing go to a site where they are likely to be exposed to infected people waiting to get tested. Ideally, rapid, surveillance level COVID-19 testing should be done. Rapid turnaround time on test results is required to effectively prevent positive individuals from infecting others as well as to conduct contact tracing.

Therefore, there is a need for rapid test result turnaround time in order to decrease viral transmission and reduce community spread of the virus. Test result turnaround time must be as rapid as possible, for example, test result turnaround time ideally should be faster than 24 hours. Furthermore, testing should be done directly at the site where it is needed, for example, schools, businesses, prisons, nursing facilities, and homeless shelters. To do so, a version of the COVID-19 nucleic acid test is needed that is accurate and rapid, can be easily adapted to a mobile vehicle space, and uses an orthogonal supply chain to the standard testing paradigm.

### SUMMARY

The present invention is directed to a method of diagnosing infection with SARS-CoV-2. The method involves first obtaining a sample from a patient, followed by the use of a mobile testing laboratory to test the sample for SARS-CoV-2 nucleic acid. The sample is tested by extracting the RNA from the patient sample, amplifying the nucleic acid using reverse transcription loop-mediated isothermal amplification (RT-LAMP), and measuring the results. Results from the RT-LAMP are obtained within 60 minutes after sample collection, where a positive result is indication of infection with SARS-CoV-2. The patient sample can be saliva or a nasopharyngeal sample.

In another aspect, results are obtained within 90 minutes after sample collection. In another aspect, the results are obtained within 4 hours after sample collection. The invention also contemplates contact tracing of the patient.

In one aspect, the mobile testing laboratory is a biosafety level 1 (BSL-1) laboratory. In another aspect, the mobile testing laboratory is a biosafety level 2 (BSL-2) laboratory.

### DESCRIPTION

The present invention involves a method for accurate, rapid testing of patient samples, in particular samples suspected of containing SARS-CoV-2. Test result turnaround time must be as rapid as possible, for example, test result turnaround time ideally should be faster than 24 hours. Furthermore, testing should be done directly at the site where it is needed, for example, schools, businesses, prisons, nursing facilities, and homeless shelters. The method involves an accurate and rapid COVID-19 nucleic acid test which can be easily adapted to a mobile vehicle space, and uses an orthogonal supply chain. Patient samples are be collected onsite, such as in the parking lot, using a variety of swab types, the sample is then immediately tested inside the vehicle, and results reported out in approximately one hour. The solution is easily scalable (ie, more vans) and can be targeted to the regions that need it as the hotspots flare up around the country (ie, drive to wherever testing is needed most).

As used herein, the following terms and variations thereof have the meanings given below, unless a different meaning is clearly intended by the context in which such term is used.

The terms “a,” “an,” and “the” as used herein are to be construed to cover both the singular and the plural unless their usage in context indicates otherwise.

As used in this disclosure, except where the context requires otherwise, the term “comprise” and variations of the term, such as “comprising,” “comprises” and “comprised” are not intended to exclude other additives, components, integers or steps. Thus, throughout this specification, unless the context requires otherwise, the words “comprise,” “comprising” and the like, are to be construed in an inclusive sense as opposed to an exclusive sense, that is to say, in the sense of “including, but not limited to.”

As used in this disclosure, except where the context requires otherwise, the method steps are not intended to be limiting nor are they intended to indicate that each step is essential to the method or that each step must occur in the order disclosed.

As used herein, “sample” refers to any sample that can be from or derived a human patient, e.g., bodily fluids (blood, nasal secretions, saliva, urine etc.), biopsy, tissue, and/or waste from the patient. Thus, tissue biopsies, stool, sputum, saliva, blood, lymph, tears, sweat, urine, nasal secretions, or the like can be used in the method, as can essentially any tissue of interest. The sample may be in a form taken directly from the patient.

The term “DNA sequence” as used herein refers to chromosomal sequence as well as to cDNA sequence.

The term “amplifying” in the context of nucleic acid amplification is any process whereby additional copies of a selected nucleic acid (or a transcribed form thereof) are produced. Typical amplification methods include various polymerase-based replication methods, including the polymerase chain reaction (PCR) methods.

An “amplicon” is an amplified nucleic acid, e.g., a nucleic acid that is produced by amplifying a template nucleic acid by any available amplification method (e.g., PCR, LCR, transcription, or the like).

A “gene” is one or more sequence(s) of nucleotides in a genome that together encode one or more expressed molecules, e.g., an RNA. The gene can include coding sequences that are transcribed into RNA which may then be translated into a polypeptide sequence, and can include associated structural or regulatory sequences that aid in replication or expression of the gene.

As used herein, an “isolated” nucleic acid or amino acid has been separated from at least one other component with which it is usually associated, such as its source or medium, another nucleic acid, another protein/polypeptide, another biological component or macromolecule or contaminant, impurity or minor component.

The term “mammal” is defined as an individual belonging to the class Mammalia and includes, without limitation, humans, domestic and farm animals, and zoo, sports, and pet animals, such as cows, horses, sheep, dogs and cats.

A “structural variant” refers to a variation of a nucleic acid sequence such as, for example, a deletion, duplication or inversion.

The term “RT-LAMP” refers to reverse transcription loop-mediated isothermal amplification. RT-LAMP can be used to amplify and detect viral RNA, in particular SARS-CoV-2 viral RNA.

Currently, reverse transcription polymerase chain reaction (RT-PCR) is used for detecting active SARS-CoV-2 infection. RT-PCR is sensitive, specific, and inexpensive. However, the scale of the pandemic has overwhelmed the capacity for such testing. Furthermore, there have been major disruptions in the supply chain at multiple points in the workflow, such as nasopharyngeal swabs, PPE, viral transport media, RNA extraction kits, pipet tips for liquid handling robots, extraction robots, and RT-PCR instruments.

RT-LAMP is a method similar to RT-PCR, and detects the virus by amplifying viral-specific nucleic acid. RT-LAMP uses oligonucleotide primers (enabling high specificity for SARS-CoV-2), polymerase enzymes, nucleotides, buffers, and a color indicator. Through a clever use of the primers, a stem-loop nucleotide structure is created that becomes self-amplifying. The assay is conducted at a constant temperature of 65 degrees Celsius. As a consequence, the thermal cycler instrument is no longer needed and can be replaced by a simple, inexpensive water bath. The assay is faster than RT-PCR, needing only 60 minutes for completion. Additionally, it has been shown RNA extraction can be skipped or greatly simplified, and that saliva/oral samples can be nearly as sensitive as nasopharyngeal swabs. The assay's final readout is colorimetric. A portable instrument such as the Genie II from Optigene (West Sussex, United Kingdom) can be used to interpret the colorimetric data as positive or negative, though the results can also be read by eye.

The present invention relates to a method for accurate, rapid testing of patient samples, in particular, samples suspected of containing SARS-CoV-2 in a mobile setting. The invention includes the availability of CLIA-certified labs inside motor vehicles, allowing for a rapid, accurate nucleic acid diagnostic testing, using an orthogonal supply chain, to the sites that need it most.

In the past, on-site or drive-thru testing sites have not been a feasible solution to testing underserved or vulnerable populations. Certain populations cannot easily travel to testing sites. Sampling these vulnerable populations where they reside is a potential solution, but there is an additional delay in obtaining the results due to the time it takes to get the sample to a central testing facility. The present invention allows for direct testing of the populations most in need of testing, such as, for example, prisons, nursing facilities, homeless shelters, schools, businesses, farms, and factories.

The samples can be collected in, for example, a parking lot. Patients collect their samples by, for example, swabbing the inside of their mouths under supervision by staff members. Importantly, since collecting oral samples is simple, a wide variety of swab types can be used. After collection, the patient places the swab into a tube with viral-inactivating buffer and closes the cap. The patient will then place the tube in a plastic bag and drop it within a collection bin. At the same time, a questionnaire can be administered to collect relevant information for contact tracing, another major pain point throughout the pandemic. The test is then run inside the mobile lab, giving rapid results. The results are ready to send to the facility and the Department of Public Health, and immediate action can be taken to quarantine positive cases. It is contemplated that more sophisticated sites (e.g., hospitals, nursing homes, prisons) may be able to collect patient samples prior to onsite arrival of the mobile lab.

The mobile lab used in the method of the invention has, at a minimum, a power source, one or more water baths, a freezer, and a laptop computer or printer to output results. Each mobile lab requires two staff members, namely a clinical laboratory scientist and an assistant. The mobile labs are cleaned and restocked daily.

In one aspect, the mobile lab can make 5 stops per day, processing 100-500 samples per stop. It is contemplated that with 10 mobile lab vehicles, 5,000-25,000 samples could be tested onsite per day, prior to scaling up. Alternatively, a mobile lab could stay at one larger site (e.g., prison) all day and be outfitted with additional water bath instruments and staff to process 1,500-2,500 samples per day onsite, prior to scaling up.

There are tests that provide rapid results, such as the Abbott ID Now test, which are point-of-care devices that detect viral nucleic acid and provides results as soon as 15 minutes after administration. However, these devices can only process two samples per run. Furthermore, there currently are not enough instruments; consequently, point-of-care devices may be best suited for lower throughput settings, such as a doctor's office. There are other point-of-care devices that use antigen-detecting chemistry to generate rapid results. However, only two have received an EUA from the FDA at this time. The lower sensitivity of these devices has prevented them from widespread use, though there are several groups working to develop better versions.

The present invention has many advantages over the current rapid tests. First, the testing method of the present invention has significantly higher sensitivity and throughput. This distinction is important: the test of the invention is not comparable to the lower-sensitivity, point-of-care tests deployed elsewhere but represents a robust, accurate assay performed by a clinical laboratory scientist in a CLIA-certified lab that happens to be mobile.

It is contemplated that the mobile lab of the invention can perform any viral diagnostic assay that is currently commercially available, or a viral diagnostic assay that is available in the future. This includes COVID-19 genetic test using saliva as sample type (1-hour turn-around time, tested onsite in vans), COVID-19 genetic test using nasal swab (6-hour turn-around time), and COVID-19 serology test (ELISA for either IgG or IgM, and titer) (3-hour turn-around time).

It is further contemplated that contact tracing of individuals testing positive for SARS-CoV-2 can be performed. The effectiveness of contact tracing depends strongly on getting fast testing results. This gets people into quarantine while they are still contagious. This has been a major challenge throughout the pandemic; by the time the result comes back positive, the infected individual is likely already past peak-contagiousness. Basic contact information plus a list of recent contacts for each patient can be collected by staff while the technicians are processing the sample. In the case of a positive result, the patient, their provider, and the Department of Public Health can be notified immediately. Having a list of recent contacts created at the time of testing should facilitate better memory recall from the patient since less time has elapsed.

### Example

Oral samples were taken from patients and donors using a Puritan Purflock ultra-flocked swab, (Puritan Medical Products, Guilford, Me.). After collection, the swabs were placed in viral inactivation media DNA/RNA Shield (Zymo Research, Irvine, Calif.) per the manufacture's protocol. Total RNA was then extracted using a Quick-DNA/RNA Viral Mag Bead RNA extraction kit (Zymo Research, Irvine, Calif.). RT-LAMP reactions were setup using the LAMP kit (New England Biolabs, Ipswich, Mass.). Primers specific for SARS-CoV-2 were used; in this case, two sets of primers were used, one set that targeted the envelope small membrane protein (E gene) and one set targeted the nucleocapsid (N gene). Control primers were also used in a parallel reaction to target human genes (RNaseP and rActin) to ensure proper patient sampling. After the 30-minute incubation at a constant 65° C., the results from the RT-LAMP reactions were read on a standard plate reader (BioTek, Winooski, Vt.) for either fluorescent or colorimetric reactions, or evaluated manually by eye in the case of colorimetric reactions. The results of the assay showed, 25 out of 25 normal donors tested negative while 10 out of 10 COVID-19 positive control clinical samples tested positive.

TABLE 1

| Primer Name | SEQ ID | Sequence |
|---|---|---|
| E1-FIP | SEQ ID No: 1 | ACCACGAAAGCAAGAAAAAGAAGTTCGTTTCGGAAGAGACAG |
| E1-BIP | SEQ ID No: 2 | TTGCTAGTTACACTAGCCATCCTTAGGTTTTACAAGACTCACGT |
| E1-F3 | SEQ ID No: 3 | TGAGTACGAACTTATGTACTCAT |
| E1-B3 | SEQ ID No: 4 | TTCAGATTTTTAACACGAGAGT |
| E1-LF | SEQ ID No: 5 | CGCTATTAACTATTAACG |
| E1-LB | SEQ ID No: 6 | GCGCTTCGATTGTGTGCGT |
| N2-FIP | SEQ ID No: 7 | TTCCGAAGAACGCTGAAGCGGAACTGATTACAAACATTGGCC |
| N2-BIP | SEQ ID No: 8 | CGCATTGGCATGGAAGTCACAATTTGATGGCACCTGTGTA |
| N2-F3 | SEQ ID No: 9 | ACCAGGAACTAATCAGACAAG |
| N2-B3 | SEQ ID No: 10 | GACTTGATCTTTGAAATTTGGATCT |
| N2-LF | SEQ ID No: 11 | GGGGGCAAATTGTGCAATTTG |
| N2-LB | SEQ ID No: 12 | CTTCGGGAACGTGGTTGACC |
| ACTB-FIP | SEQ ID No: 13 | GAGCCACACGCAGCTCATTGTATCACCAACTGGGACGACA |
| ACTB-BIP | SEQ ID No: 14 | CTGAACCCCAAGGCCAACCGGCTGGGGTGTTGAAGGTC |
| ACTB-F3 | SEQ ID No: 15 | AGTACCCCATCGAGCACG |
| ACTB-B3 | SEQ ID No: 16 | AGCCTGGATAGCAACGTACA |
| ACTB-LF | SEQ ID No: 17 | TGTGGTGCCAGATTTTCTCCA |
| ACTB-LB | SEQ ID No: 18 | CGAGAAGATGACCCAGATCATGT |

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference in their entirety.

What is claimed is:

1. A method of diagnosing infection with SARS-CoV-2, the method comprising the steps of:
a. obtaining a sample from a patient;
b. using a mobile testing laboratory to test the sample for SARS-CoV-2 RNA, wherein the testing comprises the steps of:
i. extracting RNA from the patient sample;
ii. amplifying the RNA using reverse transcription loop-mediated isothermal amplification (RT-LAMP);
iii. measuring the results of the RT-LAMP; and
c. obtaining the results of the RT-LAMP within 60 minutes after sample collection, wherein a positive result is indication of infection with SARS-CoV-2.

2. The method of claim 1, further comprising contact tracing of the patient.

3. The method of claim 1, wherein the results are obtained within 90 minutes after sample collection.

4. The method of claim 3, further comprising contact tracing of the patient.

5. The method of claim 1, wherein the results are obtained within 4 hours after sample collection.

6. The method of claim 5, further comprising contact tracing of the patient.

7. The method of claim 1, wherein the mobile testing laboratory is a biosafety level 1 (BSL-1) laboratory.

8. The method of claim 1, wherein the mobile testing laboratory is a biosafety level 2 (BSL-2) laboratory.

9. The method of claim 1, wherein the patient sample is saliva.

10. The method of claim 1, wherein the patient sample is a nasopharyngeal sample.


SEQUENCE LISTING (18 sequences)

SEQ ID NO: 1 - DNA, 42 bp, Artificial Sequence
  Primer
accacgaaagcaagaaaaagaagttcgtttcggaagagacag

SEQ ID NO: 2 - DNA, 44 bp, Artificial Sequence
  Primer
ttgctagttacactagccatccttaggttttacaagactcacgt

SEQ ID NO: 3 - DNA, 23 bp, Artificial Sequence
  Primer
tgagtacgaacttatgtactcat

SEQ ID NO: 4 - DNA, 22 bp, Artificial Sequence
  Primer
ttcagatttttaacacgagagt

SEQ ID NO: 5 - DNA, 18 bp, Artificial Sequence
  Primer
cgctattaactattaacg

SEQ ID NO: 6 - DNA, 19 bp, Artificial Sequence
  Primer
gcgcttcgattgtgtgcgt

SEQ ID NO: 7 - DNA, 42 bp, Artificial Sequence
  Primer
ttccgaagaacgctgaagcggaactgattacaaacattggcc

SEQ ID NO: 8 - DNA, 40 bp, Artificial Sequence
  Primer
cgcattggcatggaagtcacaatttgatggcacctgtgta

SEQ ID NO: 9 - DNA, 21 bp, Artificial Sequence
  Primer
accaggaactaatcagacaag

SEQ ID NO: 10 - DNA, 25 bp, Artificial Sequence
  Primer
gacttgatctttgaaatttggatct

SEQ ID NO: 11 - DNA, 21 bp, Artificial Sequence
  Primer
gggggcaaattgtgcaatttg

SEQ ID NO: 12 - DNA, 20 bp, Artificial Sequence
  Primer
cttcgggaacgtggttgacc

SEQ ID NO: 13 - DNA, 40 bp, Artificial Sequence
  Primer
gagccacacgcagctcattgtatcaccaactgggacgaca

SEQ ID NO: 14 - DNA, 38 bp, Artificial Sequence
  Primer
ctgaaccccaaggccaaccggctggggtgttgaaggtc

SEQ ID NO: 15 - DNA, 18 bp, Artificial Sequence
  Primer
agtaccccatcgagcacg

SEQ ID NO: 16 - DNA, 20 bp, Artificial Sequence
  Primer
agcctggatagcaacgtaca

SEQ ID NO: 17 - DNA, 21 bp, Artificial Sequence
  Primer
tgtggtgccagattttctcca

SEQ ID NO: 18 - DNA, 23 bp, Artificial Sequence
  Primer
cgagaagatgacccagatcatgt